SEQUENCE LISTING (12 sequences)

SEQ ID NO: 1 - DNA, 576 bp, Homo sapiens
atgcatgacagtaacaatgtggagaaagacattacaccatctgaattgcctgcaaacccaggttgtctgcattcaaaagagcattctattaaagctaccttaatttggcgcttatttttcttaatcatgtttctgacaatcatagtgtgtggaatggttgctgctttaagcgcaataagagctaactgccatcaagagccatcagtatgtcttcaagctgcatgcccagaaagctggattggttttcaaagaaagtgtttctatttttctgatgacaccaagaactggacatcaagtcagaggttttgtgactcacaagatgctgatcttgctcaggttgaaagcttccaggaactgaatttcctgttgagatataaaggcccatctgatcactggattgggctgagcagagaacaaggccaaccatggaaatggataaatggtactgaatggacaagacagtttcctatcctgggagcaggagagtgtgcctatttgaatgacaaaggtgccagtagtgccaggcactacacagagaggaagtggatttgttccaaatcagatatacatgtctag

SEQ ID NO: 2 - protein, 191 aa, Homo sapiens
MHDSNNVEKDITPSELPANPGCLHSKEHSIKATLIWRLFFLIMFLTIIVCGMVAALSAIRANCHQEPSVCLQAACPESWIGFQRKCFYFSDDTKNWTSSQRFCDSQDADLAQVESFQELNFLLRYKGPSDHWIGLSREQGQPWKWINGTEWTRQFPILGAGECAYLNDKGASSARHYTERKWICSKSDIHV

SEQ ID NO: 3 - DNA, 399 bp, Artificial sequence
  Description of Artificial Sequence Synthetic LLT1 fragment
agagctaactgccatcaagagccatcagtatgtcttcaagctgcatgcccagaaagctggattggttttcaaagaaagtgtttctatttttctgatgacaccaagaactggacatcaagtcagaggttttgtgactcacaagatgctgatcttgctcaggttgaaagcttccaggaactgaatttcctgttgagatataaaggcccatctgatcactggattgggctgagcagagaacaaggccaaccatggaaatggataaatggtactgaatggacaagacagtttcctatcctgggagcaggagagtgtgcctatttgaatgacaaaggtgccagtagtgccaggcactacacagagaggaagtggatttgttccaaatcagatatacatgtctag

SEQ ID NO: 4 - protein, 132 aa, Artificial sequence
  Description of Artificial Sequence Synthetic LLT1 fragment
RANCHQEPSVCLQAACPESWIGFQRKCFYFSDDTKNWTSSQRFCDSQDADLAQVESFQELNFLLRYKGPSDHWIGLSREQGQPWKWINGTEWTRQFPILGAGECAYLNDKGASSARHYTERKWICSKSDIHV

SEQ ID NO: 5 - DNA, 678 bp, Homo sapiens
atggaccaacaagcaatatatgctgagttaaacttacccacagactcaggcccagaaagttcttcaccttcatctcttcctcgggatgtctgtcagggttcaccttggcatcaatttgccctgaaacttagctgtgctgggattattctccttgtcttggttgttactgggttgagtgtttcagtgacatccttaatacagaaatcatcaatagaaaaatgcagtgtggacattcaacagagcaggaataaaacaacagagagaccgggtctcttaaactgcccaatatattggcagcaactccgagagaaatgcttgttattttctcacactgtcaacccttggaataacagtctagctgattgttccaccaaagaatccagcctgctgcttattcgagataaggatgaattgatacacacacagaacctgatacgtgacaaagcaattctgttttggattggattaaatttttcattatcagaaaagaactggaagtggataaacggctcttttttaaattctaatgacttagaaattagaggtgatgctaaagaaaacagctgtatttccatctcacagacatctgtgtattctgagtactgtagtacagaaatcagatggatctgccaaaaagaactaacacctgtgagaaataaagtgtatcctgactcttga

SEQ ID NO: 6 - protein, 225 aa, Homo sapiens
MDQQAIYAELNLPTDSGPESSSPSSLPRDVCQGSPWHQFALKLSCAGIILLVLVVTGLSVSVTSLIQKSSIEKCSVDIQQSRNKTTERPGLLNCPIYWQQLREKCLLFSHTVNPWNNSLADCSTKESSLLLIRDKDELIHTQNLIRDKAILFWIGLNFSLSEKNWKWINGSFLNSNDLEIRGDAKENSCISISQTSVYSEYCSTEIRWICQKELTPVRNKVYPDS

SEQ ID NO: 7 - DNA, 480 bp, Artificial sequence
  Description of Artificial Sequence Synthetic CD161 fragment
cagaaatcatcaatagaaaaatgcagtgtggacattcaacagagcaggaataaaacaacagagagaccgggtctcttaaactgcccaatatattggcagcaactccgagagaaatgcttgttattttctcacactgtcaacccttggaataacagtctagctgattgttccaccaaagaatccagcctgctgcttattcgagataaggatgaattgatacacacacagaacctgatacgtgacaaagcaattctgttttggattggattaaatttttcattatcagaaaagaactggaagtggataaacggctcttttttaaattctaatgacttagaaattagaggtgatgctaaagaaaacagctgtatttccatctcacagacatctgtgtattctgagtactgtagtacagaaatcagatggatctgccaaaaagaactaacacctgtgagaaataaagtgtatcctgactcttga

SEQ ID NO: 8 - protein, 159 aa, Artificial sequence
  Description of Artificial Sequence Synthetic CD161 fragment
QKSSIEKCSVDIQQSRNKTTERPGLLNCPIYWQQLREKCLLFSHTVNPWNNSLADCSTKESSLLLIRDKDELIHTQNLIRDKAILFWIGLNFSLSEKNWKWINGSFLNSNDLEIRGDAKENSCISISQTSVYSEYCSTEIRWICQKELTPVRNKVYPDS

SEQ ID NO: 9 - DNA, 22 bp, Artificial sequence
  Description of Artificial Sequence Synthetic PCR primer
atgcatgacagtaacaatgtgg

SEQ ID NO: 10 - DNA, 20 bp, Artificial sequence
  Description of Artificial Sequence Synthetic PCR primer
tagttggggctttgctgtaa

SEQ ID NO: 11 - DNA, 34 bp, Artificial sequence
  Description of Artificial Sequence Synthetic PCR primer
aaggggatccgagagctaactgccatcaagagcc

SEQ ID NO: 12 - DNA, 36 bp, Artificial sequence
  Description of Artificial Sequence Synthetic PCR primer
ctcgagcggccgcctagacatgtatatctgatttgg

(19) United States
(12) Patent Application Publication
(10) Pub. No.: US 2009/0074756 A1
(43) Pub. Date: Mar. 19, 2009

(54) USE OF LLT1 AND/OR CD161 FOR MODULATTING THE ACTIVITY OF CELLS OF THE IMMUNE SYSTEM

(76) Inventors: Veronique Marie Braud, Grasse (FR); Hatice Josso-Aldemir, Antibes-Juan Les Pins (FR); Virginie Prod' Homme, La Baule-Escoublac (FR)

Correspondence Address: YOUNG & THOMPSON, 209 Madison Street, Suite 500, ALEXANDRIA, VA, 22314, US

(21) Appl. No.: 11/658,663

(22) PCT Filed: Jul. 27, 2005
(86) PCT No.: PCT/EP05/08146

§ 371 Date: May 15, 2007

(30) Foreign Application Priority Data
Jul. 27, 2004 (EP) 04291913.4

(51) Int. Cl. A61K 39/395; C07K 14/00; A61K 31/7088; G01N 33/566; A61P 43/00; C12Q 1/68; C12N 5/06; A61K 38/00

(52) U.S. Cl. 424/133.1; 530/350; 514/12; 424/172.1; 514/44; 435/375; 436/501; 435/29

(57) ABSTRACT

The present invention relates to a method for modulating in vivo or in vitro the activity of cells expressing CD161 and/or LLT1, characterized in that said cells are contacted respectively with LLT1 or fragments thereof, and/or with or fragments thereof.

The present invention relates to a method for modulating the activity of cells of the immune system.

Natural killer cells and T cells are key effectors of immune responses. A panel of receptors and costimulatory molecules modulates their activity. T-cells become activated by engagement of their clonotypic T-cell antigen receptor (TCR)-CD3 complexes by specific MHC class I or II-peptide complexes and by engagement of costimulatory receptors by their ligands expressed on professional antigen-presenting cells. The antigen activation threshold can be modulated by NK receptors expressed on their cell surface.

As opposed to T cells, NK cell recognition of targets is not antigen specific and NK cells are capable of killing target cells without prior sensitization. NK cell activity is regulated by a balance between activating and inhibitory signals triggered by engagement of NK cell receptors with their ligands. NK cells generally bind and kill target cells which are deficient in the expression of MHC class I proteins (“missing self” hypothesis) (Llunggren et al, Immunol. Today. 11:237-244, 1990). This is because NK cells express NK cell inhibitory receptors that recognizes MHC class I molecules. They include Killer cell Ig-like Receptors (KIR) interacting with groups of classical MHC class I molecules, CD94/NKG2A recognizing non classical HLA-E molecule and Ig-like transcript 2 (ILT-2) interacting with most if not all MHC class I proteins (Moretta et al, Annu. Rev. Immunol. 19:197-223, 2001). Activation and lysis of target cells is triggered mainly by Natural Cytotoxicity Receptors (NCR) and NKG2D. But a number of other activating receptors or costimulatory molecules also modulate NK cell activation. Only when all receptors and costimulatory molecules and their respective ligands are identified, will it be possible to fully understand the regulation of NK cell activity and their function.

Human Lectin-like transcript 1 (LLT1) has an extracellular domain with similarity to the C-type lectin like domains shared with other NK cell receptors including CD161 (also named NKR-P1A). LLT1 was cloned in 1999 (Genebank accession number AF133299) and is localized to the NK gene complex on human chromosome 12 in between NKR-P1A and CD69 (Boles et al, Immunogenetics. 50:1-7, 1999). Recently, a monoclonal antibody L9.7 (mAb) has been described to specifically recognize LLT1 and to induce IFN-γ cytokine production but not cytotoxicity in YT cells, a human NK cell line but also in resting NK cells freshly isolated from peripheral blood and IL-2 activated NK cells (Mathew P A et al, Mol. Immunol. 40:1157, 2004). L9.7 mAb recognizes most cells from peripheral blood mononuclear cells including NK cells (4-10%), T cells (5-14%), B cells (64-73%) and monocytes (100%). LLT1 is expressed on the cell surface as a homodimer.

CD161 (also named NKR-P1A) is also a C-type lectin expressed by natural killer cells and subsets of T cells, including TCR αβ and γδ T cells, invariant CD1d-restricted Vα24⁺ NKT and CD2⁻CD3/TCR⁻ thymocytes (Lanier et al, J. Immunol. 153:2417-2428, 1994; Poggi et al, Eur. J. Immunol. 26:1266-1272, 1996; Exley et al, J. Exp. Med. 188:867-876, 1998). CD161 is a disulfide-bond homodimer. Anti-CD161 mAbs have been reported to inhibit the NK cell-mediated lysis of some Fcγ receptor⁺ tumour target cells (Lanier et al, J. Immunol. 153:2417-2428, 1994; Poggi et al, Eur. J. Immunol. 28:1611-1616, 1998). CD161 was also shown to act as a costimulatory molecule on CD1d-restricted Vα24⁺ NKT cells, mediating both proliferation and cytokine secretion (Exley et al, J. Exp. Med., 188:867-876, 1998). So far, no ligand has been identified for CD161. It was reported that CD161 binds carbohydrates but the authors later retracted the article. Expression of CD161 on the cell surface is upregulated by IL-12 cytokine (Poggi et al, Eur. J. Immunol. 28:1611-1616, 1998). CD161 has also been reported to be involved in transendothelial migration (Poggi et al, Eur. J. Immuol. 27:2345-2350, 1997). It may upregulate LFA1 Mg²⁺ binding site and β1 and β2 (CD29) integrin at the cell surface.

The present invention arises from the unexpected finding made by the Inventors, that CD161 expressing cells could be specifically linked to LLT1.

Thus, an object of the present invention is to provide a new interaction between two surface receptors of cells of the immune system, namely CD161 and LLT1.

Another object of the invention is to use this interaction to modulate the activity of cells of the immune system.

A further object of the invention is to provide pharmaceutical compositions comprising compounds liable to mimic and/or to block the above-defined interaction.

The present invention relates to a method for modulating in vivo or in vitro the activity of cells expressing CD161 and/or LLT1, characterized in that said cells are contacted respectively with LLT1 or fragments thereof, and/or with CD161 or fragments thereof.

As intended herein “modulating the activity of cells” relates to the activation or to the inhibition of the activity of said cells.

The activity of given cells can be measured according to methods well known to the man skilled in the art. Such methods notably involve measuring the migration, the proliferation, the cytokine production, or the cytotoxicity of said cells.

The present invention also relates to a method as defined above, for modulating in vivo or in vitro the activity of cells expressing CD161 and/or LLT1, characterized in that said cells are contacted respectively with LLT1 or fragments thereof, and/or with CD161 or fragments thereof, the contacting of said cells with LLT1 or fragments thereof, and/or with CD161 or fragments thereof comprising respectively an interaction between LLT1, or fragments thereof, with CD161 expressed by said cells, or an interaction between CD161, or fragments thereof, with LLT1 expressed by said cells.

As intended herein, the expression “comprising an interaction” means that the contacting of cells expressing CD161 and/or LLT1 respectively with LLT1 or fragments thereof, and/or with CD161 or fragments thereof, entails the mutual binding of CD161, or fragments thereof, and of LLT1, or fragments thereof.

According to a preferred embodiment of the above-defined method, the CD161 expressing cells are NK cells, CD1d restricted Vα24⁺ NKT cells, or T cells, such as αβ T cells or γδ T cells.

According to another preferred embodiment of the above-defined method, when the CD161 expressing cells are NK cells, the contacting of said cells with LLT1 or fragments thereof leads to the inhibition of said cells.

Under certain conditions, the contacting of CD161 expressing NK cells with LLT1 or fragments thereof also leads to the activation of said cells.

In another preferred embodiment of the above-defined method, when the CD161 expressing cells are T cells or CD1d restricted Vα24+NKT cells, the contacting of said cells with LLT1 or fragments thereof leads to the activation of said cells.

Under certain conditions, the contacting of CD161 expressing T cells or Vα24⁺ NKT cells with LLT1 or fragments thereof also leads to the inhibition of said cells.

In yet another preferred embodiment of the above-defined method, the CD161 expressing cells are contacted with:

a human LLT1, in particular as represented by SEQ ID NO: 2, or

a fragment of said human LLT1 corresponding to the extracellular domains of LLT1, in particular as represented by SEQ ID NO: 4.

Advantageously, extracellular domains of LLT1 are soluble by themselves.

According to a particular embodiment of the above-defined method, LLT1, or fragments thereof, are presented at the surface of biological membranes, and in particular at the surface of cells, are used as soluble molecules, or are immobilized on a support.

By “biological membrane” is meant any membrane constituted of amphiphatic lipids, such as glycerophospholipids.

By “support”, is meant any solid material onto which proteins can be attached, either directly or by means of a suitable linker molecule, according to methods well-known to the man skilled in the art.

According to another particular embodiment of the above-defined method, the LLT1 fragment is part of a compound comprising one or more LLT1 fragments.

Such LLT1 comprising compounds are defined hereafter. Advantageously, the presence of more than one LLT1 fragments in said compounds increases the potency of said compounds to modulate the activity of CD161 expressing cells.

In a particularly preferred embodiment of the above-defined method, CD161 expressing cells express the human CD161 receptor, in particular as represented by SEQ ID NO: 6.

The present invention also relates to a method as defined above, characterized in that the LLT1 expressing cells are NK cells, T cells, dendritic cells, macrophages, monocytes or B cells.

According to a preferred embodiment of the above-defined method the contacting of LLT1 expressing cells with CD161 or fragments thereof leads to the activation of said cells.

Under certain conditions the contacting of LLT1 expressing cells with CD161 or fragments thereof also leads to the inhibition of said cells.

According to another preferred embodiment of the above-defined method the LLT1 expressing cells are contacted with:

a human CD161, in particular as represented by SEQ ID NO: 6, or

a fragment of said human CD161 corresponding to the extracellular domains of CD161, in particular as represented by SEQ ID NO: 8.

According to another preferred embodiment of the above-defined method CD161, or fragments thereof, are presented at the surface of biological membranes, and in particular at the surface of cells, are used as soluble molecules, or are immobilized on a support.

In another preferred embodiment of the above-defined method the CD161 fragment is part of a compound comprising one or more CD161 fragments.

Advantageously, the presence of more than one CD161 fragments in said compound increases the potency of said compound to modulate the activity of LLT1 expressing cells.

In yet another preferred embodiment of the above-defined method, LLT1 expressing cells express the human LLT1 receptor, in particular as represented by SEQ ID NO: 2.

According to a particularly preferred embodiment of the above-defined method, LLT1 or fragments thereof, or CD161 or fragments thereof, may be modified by the insertion, the deletion or the substitution of at least one amino acid, provided that said modifications result in an improved CD161-LLT1 interaction, such as an enhanced binding capability or an improved stability. In particular, modified LLT1 or CD161 having an improved interaction stability at elevated temperatures, such as temperatures over 4° C., preferably over 20° C., and more preferably at approximately 37° C., are particularly preferred in the frame of the present invention.

The present invention also relates to a method for identifying, purifying, inactivating or destroying CD161 and/or LLT1 expressing cells, characterized in that said cells are contacted respectively with LLT1 or fragments thereof, and/or with CD161 or fragments thereof.

Accordingly, for identifying CD161 and/or LLT1 expressing cells, respectively LLT1 or CD161 will be advantageously linked to marker moieties such as fluorophores or haptens.

For purifying methods, LLT1 or fragments thereof, or CD161 or fragments thereof will be advantageously linked to a solid support or to marker moieties, so that the respective association of CD161 or LLT1 expressing cells leads to a complex which can be easily retrieved from a medium containing unpurified CD161 or LLT1 expressing cells.

For inactivating or destroying CD161 and/or LLT1 expressing cells, LLT1 or fragments thereof, or CD161 or fragments thereof will be advantageously linked to antibodies, or to toxic or cytolytic moieties, such as toxins or radioactive molecules. For destroying CD161 and/or LLT1 expressing cells, LLT1 or fragments thereof, or CD161 or fragments thereof will be advantageously linked to marker moieties such as fluorophores to visualize the cells and then destroy them by use of a laser.

The present invention also relates to a method for screening compounds liable to be used for the treatment of cancers, infectious diseases, such as viral, bacterial, or parasitic diseases, autoimmune diseases, inflammatory diseases, allergic reactions, pregnancy failures, or organ and bone marrow transplantation rejection, characterized in that the compounds liable to be used for the treatment of the above mentioned pathologies are selected on their properties of inhibiting the binding between CD161 or fragments thereof, and LLT1 or fragments thereof.

As intended herein cancers notably comprise leukaemia, lymphoproliferative cancers, cervical cancers, prostate cancers, lung cancers, breast cancers, and melanoma. Infectious diseases comprise in particular HIV infection, human Cytomegalovirus infection, Hepatitis B and C infection, Ebola virus infection, Dengue, Yellow fever, Listeriosis, Tuberculosis, Cholera, Malaria, Leishmaniasis, and Trypanosoma infection. Autoimmune, inflammatory and allergic diseases notably comprise type I diabetes, rheumatoid arthritis, Lupus, inflammatory bowel diseases, such as celiac disease or Crohn disease, psoriasis, and multiple sclerosis. Pregnancy failures notably relate to implantation disorders. Organ transplantation in particular relates to xenotransplantations.

Several studies show that CD161 expression is either increased or reduced in cancerous contexts:

in NK-type lymphoproliferative disease of granular lymphocytes (NK-LDGL), the frequency of CD161 expressing NK cells in PBMCs is significantly reduced compared to healthy donors (p<0.0001) (Pascal et al. (2004) Eur J Immunol 34:2930-2940);

T lymphocytes infiltrating human prostate carcinoma express high level of CD161 (Bronte et al. (2005) J Exp Med 201:1257-1268);

Tumor-infiltrating lymphocytes (TILs) in human cervical cancer express up-regulated CD94/NKG2A inhibitory receptor and concomitantly lower levels of CD161. (Sheu et al. (2005) Cancer Res 65:2921-2929).

Thus, LLT1/CD161 interaction is probably modulating anti-tumor immune responses. As such blocking the interaction between LLT1 and CD161 should enhance NK cell mediated tumor rejection, whereas promoting the interaction should enhance CD8+ T cell mediated rejection.

Further, recent data have shown the efficiency of alloreactive NK cells in the cure of leukaemia. A particularly favourable outcome was observed in a group of patients who received a haploidentical bone-marrow transplant, that is when the bone-marrow donor and recipient are identical for one MHC class I haplotype and fully mismatched for the other. In this allogeneic setting, a fraction of NK cells of the donor express KIR that fail to recognize one or more MHC class I of the host. As KIR interaction with MHC class I induces an inhibitory signal, these cells are not inhibited and can kill residual leukemic cells. At the same time, a markedly reduced incidence of graft-versus-host disease is observed due to the killing of recipient dendritic cells by these alloreactive NK cells (Ruggeri et al. (1999) Blood 94:333-9; Ruggeri et al. (2002) Science 295:2097-100). So blocking interaction of LLT1 with inhibitory CD161 on NK cells should have the same effect.

As regards infections, it is clear that NK cells play an important role in immune responses to infections, either directly acting during the initial phase called innate immune response, but also in the second phase or at least in the initiation of the second phase called adaptive immune responses. In this second phase, it is also important to consider the manipulation of LLT1 or CD161 not only on NK cells but also on T cells.

Thus, an impairment of CD161 expression on NK and T cells in patients infected with HIV-1 has been reported (Alter et al. (2004) J Immunol 173:5305-5311; Jacobs et al. (2004) Clin Immunol 24:281-286).

Further, the modulation of LLT1 and CD161 interaction should also have an impact in human Cytomegalovirus infection, Hepatitis B and C virus infection, Ebola virus infection, Dengue, Yellow fever, Listeriosis, Tuberculosis, Cholera, Malaria, Leishmaniasis, and Trypanosoma infection.

NK cells and T cells also play a major role in autoimmunity:

In diabetes, for example, there is evidence for a role of NK cells and NK receptors on T cells regulating diabetes. Autoimmune (type 1) diabetes results from a loss of beta cells that is mediated by self-reactive T cells. It has been reported that NK cells mediate the protective effects of CFA (Complete Freund's Adjuvant) possibly through the down-regulation of autoreactive CTL and stimulation of NK cells, which represents a novel approach to the prevention of autoimmune diabetes (Lee et al. (2004) J. Immunol. 172:937-42). Further, the NK cell NKG2D receptor is an activating receptor and represents a new therapeutic target for autoimmune type I diabetes (Ogasawara et al. (2004) Immunity 20:757-67). It thus can be postulated that modulating LLT1/CD161 interaction should improve the outcome by inhibiting autoagressive T cells or modulating the activity of NK cells.

In rheumatoid arthritis (RA), a subset of NK cells is expanded within inflamed joints. The functional properties of these NK cells renders them good candidates for a role in interacting with the macrophage/monocyte population within the joint, thus amplifying the production of proinflammatory cytoidnes (Dalbeth & Callan (2002) Arthritis Rheum 46:1763-72). Further, expansion of an unusual subset of CD4⁺CD28⁻ T cells that infiltrates the tissue lesions has been shown to occur in RA. These cells are KIR/KAR+, CD94−, CD161+. CD4+CD161+ T cells are present in follicular microstructures typical of rheumatoid synovitis, which links them to involvement in the disease. (Warington et al. (2001) Arthritis Rheum 44:13-20). So it can be postulated that modulating LLT1/CD161 interaction should improve the outcome by inhibiting autoagressive T cells and NK cells.

Celiac disease (an inflammatory bowel disease) is a gluten-induced enteropathy characterized by the presence of gliadin-specific CD4(+) T cells in the lamina propria and by a prominent intraepithelial T-cell infiltration of unknown mechanism. In active celiac disease, there is a specific and selective increase of IELs (IntraEpithelial Lymphocytes) expressing CD94, but IELs also express CD161 both in the disease and in healthy controls. CD161+ T cells are abundant in human intestinal epithelium and most likely play a role in local immunity. Modulation of the activity of these CD161-expressing cells should therefore provide for a new therapeutic strategy against this pathology.

Psoriasis is a Th1 disease. It is triggered when activated immunocytes infiltrate the skin, subsequently inducing prominent epidermal thickening and angiogenesis. Engrafting human skin onto SCID mice is a good animal model for psoriasis. When activated CD4+ T cells but not CD8+ T cells were injected into engrafted pre-psoriasis skin, typical psoriatic plaques were created (Nickoloff and Wrone-Smith (1999) Am J Pathol 155:145-158). The blood-derived psoriatic immunocytes in the skin graft expressed CD161. In this case modulation of the activity of these CD161-expressing cells should also provide for a new therapeutic strategy against this pathology.

CD161 expressing cells seem also to be involved in Crohn disease, in Lupus and in multiple sclerosis.

It has been shown that CD161 expression by CD56+ T cells was significantly increased in women with implantation failures when compared with normal controls (Ntrivalas et al. (2005) Am J Reprod Immunol 53:215-221; Yamada et al. (2004) Am J Reprod Immunol 51:241-247). It is therefore likely that a dysregulation of this mechanism could be responsible for major pregnancy failures.

In xenotransplantation, inhibition of NK-mediated immunity through the modulation of LLT1-CD161 interaction should restrict graft rejection. Further, expression of LLT1 in graft cells, such as porcine endothelial cells in case of porcine xenotransplantation, may directly protect sensitive graft cells from human NK cell-mediated xenogeneic cytotoxicity.

Accordingly, a method for screening compounds with biological activity comprises providing cells expressing CD161 and/or LLT1 receptors at their cell surface, contacting the cells respectively with LLT1 or fragments thereof, and/or with CD161 or fragments thereof in the presence of the test compound, and determining whether the presence of the compound affects the binding of LLT1 or fragments thereof, and/or with CD161 or fragments thereof to CD161 and/or LLT1 expressing cells respectively. Preferably the cells expressing CD161 and/or LLT1 receptors in the method according to this aspect of the invention do not naturally express the receptors, and most preferably they are non-human cells. The cells are preferably stable transfectants, that is to say they contain nucleic acid material expressing CD161 and/or LLT1 stably integrated into their genome.

Alternatively, CD161 and LLT1, or fragments thereof, need not necessarily be expressed at the surface of cells. They can be linked to a solid support or both be in solution.

The present invention also relates to a method for screening compounds liable to be used for the treatment of cancers, infectious diseases, such as viral, bacterial, or parasitic diseases, autoimmune diseases, inflammatory diseases, allergic reactions, pregnancy failures, or organ and bone marrow transplantation rejection, characterized in that the compounds liable to be used for the treatment of the above mentioned pathologies are selected on their properties of inducing CD161 internalization from CD161-expressing cells.

The expression “CD161 internalization from CD161-expressing cells” means that compounds are selected on their capacity of inducing downregulation of CD161 molecules which are expressed at the surface, i.e. in the plasma membrane, of said CD161-expressing cells.

Downregulation of CD161 molecules can be evidenced according to numerous methods well known to the man skilled in the art, such as those described in the Examples.

In a preferred embodiment of the above mentioned screening method, the compounds are selected:

on their properties of inhibiting the binding between CD161 or fragments thereof, and LLT1 or fragments thereof, and

on their properties of inducing CD161 internalization from CD161-expressing cells.

In a preferred embodiment of the above-mentioned screening method, the compounds to screen are antibodies, such as anti-CD161 or anti-LLT1 antibodies, or fragments thereof such as Fab, F(ab)′₂ or scFv fragments.

The antibodies to screen are in particular monoclonal antibodies and/or humanized antibodies.

In another preferred embodiment of the above-mentioned screening method the compounds to screen are aptamers.

The present invention also relates to an isolated compound comprising at least two LLT1 molecules or fragments thereof.

The expression “isolated” relates to a compound comprising at least two LLT1 molecules or fragments thereof which is not part of a biological membrane. In particular, the isolated compound according to the invention is soluble.

In such a compound, the LLT1 molecules or fragments thereof are linked together, either directly or indirectly, via linker molecules, through covalent or non-covalent bonds.

According to a preferred embodiment of the isolated LLT1 comprising compound, said compound comprises LLT1 fragments as represented by SEQ ID NO: 4.

According to a another preferred embodiment of the isolated LLT1 comprising compound, the LLT1 fragments are linked to anchoring moieties, such as a γ-immunoglobulin Fc domain or biotin, and associated together via a linker molecule which binds to said anchoring molecules, such as protein A or an avidin protein.

Anchoring molecules may also be made of, or covered with a magnetic material or a biologically inert material, such as silicon, and be in the form of beads.

According to yet another preferred embodiment of the isolated LLT1 comprising compound, said compound also comprises:

toxic or cytolytic moieties, such as toxins, radioactive molecules, recombinant active molecules or chemicals,

marker moieties, such as fluorophores, magnetic beads, or haptens.

The present invention also relates to a pharmaceutical composition comprising as active substance, a compound liable to bind to CD161 or a compound liable to bind to LLT1, in association with a pharmaceutically acceptable carrier.

According to a preferred embodiment of the invention, the above-defined pharmaceutical composition comprises as active substance, a mammalian LLT1 or fragments thereof, as compounds liable to bind to CD161, or a mammalian CD161 or fragments thereof, as compounds liable to bind to LLT1, in association with a pharmaceutically acceptable carrier.

According to another preferred embodiment of the invention, the above-defined pharmaceutical composition comprises as active substance,

a human LLT1, in particular as represented by SEQ ID NO: 2, or

a fragment of said human LLT1 corresponding to the extracellular domain of LLT1, in particular as represented by SEQ ID NO: 4

According to another preferred embodiment of the invention, the above-defined pharmaceutical composition comprises as active substance an above-defined LLT1 comprising compound.

In another preferred embodiment, the present invention relates to an above-defined pharmaceutical composition comprising as active substance:

a human CD161, in particular as represented by SEQ ID NO: 6, or

a fragment of said human CD161 corresponding to the extracellular domain of CD161, in particular as represented by SEQ ID NO: 8.

In a particularly preferred embodiment, the present invention relates to an above-defined pharmaceutical composition, comprising as active substance an anti-CD161 or anti-LLT1 antibody, in particular a humanized anti-CD161 or anti-LLT1 antibody.

Such antibodies may be monoclonal or polyclonal antibodies, which can be easily obtained according to methods well-known to the man skilled in the art. In particular, monoclonal antibodies can be produced from hybridomas. Such hybridomas can be obtained by fusion of an antibody secreting cell, such as a B cell, with an immortalized cell, according to Kohler and Milstein. Nature. 256:495-497, 1975 or to Buttin et al. Curr. Top. Microbiol. Immun. 81:27-36, 1978, for example.

As intended herein, the fragments of such antibodies, such as Fab, F(ab)′₂ or scFv fragments, are also considered as antibodies.

Humanized antibodies can be for example chimeric antibodies, in which, if appropriate, constant parts of animal antibodies, such as mouse or rabbit antibodies, are replaced by the corresponding parts of human antibodies, such as the Fc fragment for instance (Sharon et al, Nature. 309:364-367, 1984; Neuberger et al, Nature. 314:268-271, 1985). Alternatively, the complex determining region (CDR) of the animal antibodies can be grafted onto human antibodies, such as described for instance in U.S. Pat. No. 5,824,307, a process called “antibody reshaping” (Jones et al. Nature, 321:522-525, 1986). Another alternative technique is to produce human antibodies in mice using transgenic and transomic animals (Bruggemann et al, PNAS. 86:6709-6713, 1989; Richa et al, Science. 245:175-177, 1989; Capecchi, Trends in Genetics. 5:70-76, 1989).

The present invention also relates to a pharmaceutical composition comprising as active substance, single or multiple stranded nucleic acids encoding mammalian, in particular human, LLT1 or fragments thereof, or their complementary strands in the case of single stranded nucleic acids, in particular as represented respectively by SEQ ID NO: 1 and SEQ ID NO: 3, optionally comprised in a mammalian expression vector, in association with a pharmaceutically acceptable carrier.

The present invention also relates to a pharmaceutical composition comprising as active substance, single or multiple stranded nucleic acids encoding mammalian, in particular human, CD161 or fragments thereof, or their complementary strands in the case of single stranded nucleic acids, in particular as represented respectively by SEQ ID NO: 5 and SEQ ID NO: 7, optionally comprised in a mammalian expression vector, in association with a pharmaceutically acceptable carrier.

Such nucleic acids or vectors are useful for various purposes, such as:

the in vivo, ex vivo or in vitro production of the proteins they encode, i.e. LLT1 or fragments thereof and CD161 or fragments thereof;

the in vivo, ex vivo or in vitro production or delivery of antisens nucleic acids, which are liable to inhibit the expression of CD161 or LLT1;

the in vivo, ex vivo or in vitro production or delivery of interference nucleic acids, such as siRNA, which are liable to inhibit the expression of CD161 or LLT1.

The present invention also relates to the use of a compound liable to bind to CD161 or a compound liable to bind to LLT1, for the manufacture of a medicament intended for the prevention or the treatment of cancers, infectious diseases, such as viral, bacterial, or parasitic diseases, autoimmune diseases, inflammatory diseases, allergic reactions, pregnancy failures, or organ and bone marrow transplantation rejection.

In a preferred embodiment, the present invention relates to the above-defined use of a mammalian LLT1 or fragments thereof, as compounds liable to bind to CD161, or of a mammalian CD161 or fragments thereof, as compounds liable to bind to LLT1.

In another preferred embodiment, the invention relates to the above-defined use, of

a human LLT1, in particular as represented by SEQ ID NO: 2, or

a fragment of said human LLT1 corresponding to the extracellular domain of LLT1, in particular as represented by SEQ ID NO: 4.

In a particularly preferred embodiment the present invention relates to the above-defined use, of a LLT1 comprising compound as defined above.

In another embodiment, the present invention relates to the above-defined use, of:

a human CD161, in particular as represented by SEQ ID NO: 6, or

a fragment of said human CD161 corresponding to the extracellular domain of CD161, in particular as represented by SEQ ID NO: 8.

In a preferred embodiment, the invention also relates to the above-defined use, of an anti-CD161 or anti-LLT1 antibody, in particular a humanized anti-CD161 or anti-LLT1 antibody.

The present invention also relates to the use of single or multiple stranded nucleic acids encoding mammalian, in particular human, LLT1 or fragments thereof, or their complementary strands in the case of single stranded nucleic acids, in particular as represented respectively by SEQ ID NO: 1 and SEQ ID NO: 3, optionally comprised in a mammalian expression vector, for the manufacture of a medicament intended for the prevention or the treatment of cancers, infectious diseases, such as viral, bacterial, or parasitic diseases, autoimmune diseases, inflammatory diseases, allergic reactions, pregnancy failures, or organ and bone marrow transplantation rejection.

The present invention also relates to the use of single or multiple stranded nucleic acids encoding mammalian, in particular human, CD161 or fragments thereof, or their complementary strands in the case of single stranded nucleic acids, in particular as represented respectively by SEQ ID NO: 5 and SEQ ID NO: 7, optionally comprised in a mammalian expression vector, in association with a pharmaceutically acceptable carrier, for the manufacture of a medicament intended for the prevention or the treatment of cancers, infectious diseases, such as viral, bacterial, or parasitic diseases, autoimmune diseases, inflammatory diseases, allergic reactions, pregnancy failures, or organ and bone marrow transplantation rejection.

As intended herein cancers notably comprise leukaemia, lymphoproliferative cancers, cervical cancers, prostate cancers, lung cancers, breast cancers, and melanoma. Infectious diseases comprise in particular HIV infection, human Cytomegalovirus infection, Hepatitis B and C infection, Ebola virus infection, Dengue, Yellow fever, Listeriosis, Tuberculosis, Cholera, Malaria, Leishmaniasis, and Trypanosoma infection. Autoimmune, inflammatory and allergic diseases notably comprise type I diabetes, rheumatoid arthritis, Lupus, inflammatory bowel diseases, such as celiac disease or Crohn disease, psoriasis, and multiple sclerosis. Pregnancy failures notably relate to implantation disorders. Organ transplantation in particular relates to xenotransplantations.

### DESCRIPTION OF THE FIGURES

FIG. 1A and FIG. 1B

Production of Chimeric LLT1-Fc Fusion Protein

FIG. 1A: Purified LLT1-Fc fusion protein was visualized on a 10% SDS-PAGE gel using reducing and nonreducing conditions yielding a ˜55 kDa and ˜110 kDa protein respectively. FIG. 1B: Treatment of purified LLT1-Fc with peptide N-glycosidase F (PNGase F) yielded a ˜48 kDa protein.

FIG. 2

LLT1 Interacts with CD161 Receptor

LLT1 multimer were generated using LLT1-Fc dimer conjugated to protein A-biotin and streptavidin-APC. Saturating amount of purified human IgG were added to block the remaining free protein A sites. A multimer control was generated by incubating ProteinA-biotin with saturing amount of purified human IgG.

LLT1 and Ctrl multimers were used to stain HEK293T cell untransfected or transfected with CD161-pIRES2-EGFP or LLT1-pIRES2-EGFP vectors.

LLT1 multimer bound to HEK293T cells transfected with CD161 but not to HEK293T expressing LLT1 or untransfected cells. Control multimer did not bind to any of these cells.

FIG. 3A and FIG. 3B

LLT1 Multimer Staining to CD161+ Cells is Inhibited by Anti-CD161 Antibodies

FIG. 3A: LLT1 multimer was used to stain HEK293T cells transfected with CD161-pIRES2-EGFP or LLT1-pIRES2-EGFP vectors in the absence or presence of 3 μg of irrelevant isotype IgG control (DX22) or anti-CD161 antibody (DX12). Similar blocking was seen using 191B8 anti-CD161 antibody.

FIG. 3B: LLT1 multimer was used to stain HEK293T cells transfected with CD161-pIRES2-EGFP (squares) or LLT1-pIRES2-EGFP (circles) vectors in the presence of increasing concentration of irrelevant isotype IgG control (DX22) (open symbols) or anti-CD161 antibody (DX12) (closed symbols).

FIG. 4A and FIG. 4B

Interaction of Polyclonal CD161⁺ NK and T Cells with Target Cells Expressing LLT1 Results in a Selective Downregulation of CD161 on NK and T Cells

HEK293T cells and Hela cells transfected or not with LLT1 were incubated with a polyclonal human NK and T cell population for 2 and 4 hours in complete medium at 37° C. (ratio 1:1). Cells were fixed and stained with anti-CD161 antibodies to monitor CD161 level of expression at the cell surface of NK and T cells.

FIG. 4A: % of CD161⁺ NK cells incubated with the indicated targets for 2 and 4 hours.

FIG. 4B: level of CD161 expression (mean fluorescence) on NK cells incubated with the indicated targets for 2 and 4 hours.

Similar downregulation of CD161 on T cells is observed.

FIG. 5

LLT1-Induced Downregulation of CD161 is Blocked by Anti-CD161 Antibodies

Hela cells transfected or not with LLT1 were incubated with a polyclonal human NK and T cell population for 2 hours in complete medium at 37° C. (ratio 1:1) in the presence of anti-CD161 (DX12) mAb or isotype IgG control (5 μg/1 ml). Cells were fixed and stained with anti-CD161 antibody 191B8 (which recognizes a different epitope to DX12) to monitor CD161 level of expression at the cell surface of NK and T cells. % of CD161+ NK cells is shown and similar blocking is observed on T cells (data not shown). Identical blocking of LLT1-induced downregulation of CD161 is detected when anti-CD161 mAb 191B8 is used to block and anti-CD161 mAb DX12 is used to stain cells (data not shown).

FIG. 6

LLT1-Induced Downregulation of CD161 on NK and T Cells Needs Cell-to-Cell Contacts

HEK293T cells transfected or not with LLT1 were incubated either in normal wells or transwells with a polyclonal human NK and T cell population for 4 hours in complete medium at 37° C. (ratio 1:1). CD161 level of expression on NK and T cells was monitored using anti-CD161 mAb.

FIG. 7

Expression of LLT1 in HEK293T Cells Partially Inhibits Polyclonal IL-2 Activated NK Cell-Mediated Cytotoxicity.

293T cells transfected or not with LLT1 were labelled with ⁵¹chromium and incubated with polyclonal IL-2 activated NK cells for 18 hours at an E:T ratio of 20:1. Release of ⁵¹chromium was measured in the supernatant and % of specific lysis of the targets calculated.

FIG. 8

LLT1 Interaction with CD161 Results in a Downregulation of CD161 on NK Cells

FIG. 8 represents the CD161 cell surface expression (mean fluorescence intensity (MFI)) upon time on NK cells either untreated (squares) or fixed (circles) and incubated with untransfected C1R cells (white), or with CR1 cells expressing LLT1 (black). Cell surface CD161 decreased upon time on NK cells incubated with C1R-LLT1 transfectants but not when NK cells were fixed prior exposure or when they were incubated with C1R cells.

FIG. 9

LLT1-Induced Downregulation of CD161 on NK Cells Required Cell-to-Cell Contacts

FIG. 9 represents CD161 cell surface expression (vertical axis, mean fluorescence in %) on NK cells incubated with untransfected 293T cells (white squares) or with LLT1-expressing 293T cells (black squares) in settings allowing intercellular contacts or forbidding them (transwells). NK cells were incubated with 293T or 293T-LLT1 cells for 4 hr at 37° C. The mean fluorescence intensities obtained after exposure to negative control 293T cells were arbitrarily set as 100%.

FIG. 10

LLT1-Induced Downregulation of CD161 on NK Cells is not Affected by Bafilomycin and Chloroquine Treatment.

FIG. 10 represents CD161 cell surface expression (hatched bar) and total CD161 expression (black bar) (vertical axis, mean fluorescence in %) on NK cells incubated with untransfected C1R cells or LLT1 expressing C1R transfectants untreated or treated with two agents blocking intracellular degradation (chloroquine and bafilomycin A1). Treatment with DMSO 1% is the control for bafilomycin diluted in DMSO. Surface and total CD161 expression decreased after exposure of NK cells to C1R-LLT1 transfectants in the presence or absence of bafilomycin A1 and chloroquine. NK cells were preincubated with bafilomycin A1 (0.5 μM) and chloroquine (33 μM) for 1 hr at 37° C., followed by incubation for 4 hr with C1R or C1R-LLT1 cells. The mean fluorescence intensities obtained after exposure to negative control C1R cells were arbitrarily set as 100%. CD56 surface expression (white bar) and total expression (grey bar) were monitored and used as a negative control marker.

FIG. 11A and FIG. 11B

LLT1 Transcripts

FIGS. 11A and 11B represent an electrophoresis gel showing the results of RT-PCR experiments carried on various cell lines (on top) for detecting the presence of LLT1 transcripts. The GAPDH transcript is used as an internal control.

FIG. 12

LLT1 Interaction with CD161 Results in an Inhibition of NK Cell-Mediated Cytotoxicity

NK cell-mediated cytotoxicity was measured in a chromium release assay against C1R and C1R-LLT1 target cells. FIG. 12 represents the percentage of untransfected C1R cells (squares) or LLT1-expressing C1R cells (circles) lysed by NK cells, in the absence of monoclonal antibody (black squares and circles), in the presence of an irrelevant antibody (mIgG2a, grey quares and circles) or in the presence of an anti-CD161 mAb (αCD161, white squares and circles). Effector (NK cells) to Target (C1R or C1R-LLT1) ratio used were 10:1, 5:1, 1:1. Isotype control mIgG2a and anti-CD161 mAb (191B8) were used at 10 μg/ml. LLT1 expression significantly inhibited NK cell-mediated cytotoxicity which was restored by blocking anti-CD161 mAb.

FIG. 13

LLT1 Interaction with CD161 Results in an Inhibition of NK Cell-Mediated Cytotoxicity: Redirected Killing

FIG. 13 represents the percentage of untransfected FcR-bearing P815 cells (squares) or LLT1-expressing P815 cells (circles) lysed by NK cells in the presence of an irrelevant antibody (mIgG2a, dark grey quares and circles), of an anti-CD161 mAb (αCD161, light grey squares and circles), of an irrelevant antibody and an activating anti-CD16 antibody (αCD16+mIgG2a, black squares and circles), or of an anti-CD161 mAb and an activating anti-CD16 antibody (αCD16+αCD161, white squares and circles). Effector (NK cells) to Target (C1R or C1R-LLT1) ratio used were 5:1, 1:1, 0.5:1. Redirected killing could be inhibited by expression of LLT1 on the FcR expressing cell line P815 or by addition of anti-CD161 mAb together with anti-CD16 used to induce NK cell cytotoxicity.

FIG. 14

LLT1 Interaction with CD161 Results in an Inhibition of NK Cell-Mediated Cytotoxicity

LAMP-1 or CD107a is a marker of activation induced degranulation, a necessary precursor of cytolysis. FIG. 14 represents CD161 surface expression (vertical axis) versus CD107a surface expression (horizontal axis) on polyclonal NK cells incubated alone (medium), in the presence of C1R cells or in the presence of LLT1-expressing C1R cells, as measured by flow cytometry. The percentage of CD161+/CD107a+ cells is indicated. NK cells were incubated with C1R or C1R-LLT1 cells for 1 hr in the presence of anti-CD107a mAb followed by 4 hr in the presence of Brefeldin A (10 μg/nml). Effector to target ratio was 1:5. Cells were fixed and stained for surface expression of CD161.

FIG. 15

LLT1 Interaction with CD161 Inhibits IFN-γ Production by NK Cells

FIG. 15 represents IFNγ production (vertical axis) versus CD161 surface expression (horizontal axis) in polyclonal NK cells incubated alone (medium), in the presence of C1R cells, in the presence of LLT1-expressing C1R cells, or in the presence of PMA/ionomycin (positive control), as measured by flow cytometry. The percentage of IFNγ+/CD161+ cells is indicated. The polyclonal NK cell population was incubated for 4 hr at 37° C. with C1R or C1R-LLT1 target cells in the presence of brefeldin A (10 μg/ml). Effector to target ratio was 1:5. NK cells were identified by cell surface staining using CD161 and CD56 markers and IFN-γ producing cells by intracellular staining.

FIG. 16

LLT1 Interaction with CD161 Inhibits IFN-γ Production by NK Cells

FIG. 16 represents IFNγ production (vertical axis) versus CD161 surface expression (horizontal axis) in polyclonal NK cells incubated with C1R cells (upper panels) or LLT1-expressing C1R cells (lower panels) in the presence of a control antibody (+Ctrl Ig2a at 10 μg/ml) or an anti-CD161 antibody (191B8) at 1 μg/ml, 1 μg/ml or 0.1 μg/ml. The percentage of IFN-γ NK cells is indicated.

FIG. 17

Ligation of CD161 Enhances CD3 Triggered-IFN-γ Production by T Cells

A polyclonal T cell population was incubated for 4 hr at 37° C. with P815 or P815-LLT1 target cells in the presence of increasing concentrations of anti-CD3 and brefeldin A (10 μg/ml). FIG. 17 represents IFN-γ production (vertical axis) versus CD161 surface expression (horizontal axis) in polyclonal T cells incubated with P815 cells (upper panels) or LLT1-expressing P815 cells (lower panels) either without any antibody (no mAb), or in the presence of an anti-CD3 antibody at 5 μg/ml, 500 ng/ml or 50 ng/ml. Effector to target ratio was 1:2.5. T cells were identified by cell surface staining using CD3 and CD161 markers and IFN-γ producing cells by intracellular staining. The percentage of IFNγ+/T cells is indicated.

FIG. 18

Ligation of CD161 Enhances CD3 Triggered-IFN-γ Production by T Cells

A polyclonal T cell population was incubated for 4 hr at 37° C. with plate-bound anti-CD3 (500 ng/ml) alone or together with isotype control mIgG2a (5 μg/ml) or anti-CD161 191B8 (5 μg/ml) and in the presence of brefeldin A (10 μg/ml). FIG. 18 represents IFNγ production (vertical axis) versus CD161 surface expression (horizontal axis) in polyclonal T cells incubated on plate-bound anti-CD3 antibody (+αCD3), anti-CD3 antibody and a control antibody (+αCD3+αIgG2a) and anti-CD3 antibody and a an anti-CD161 antibody (+αCD3+αCD161). T cells were identified by cell surface staining using CD3 and CD161 markers and IFN-γ producing cells by intracellular staining. The percentage of IFN-γ+T cells is indicated.

FIG. 19

CD161 Cell Surface Level of Expression is Upregulated with Increasing Concentrations of IL-12

FIG. 19 represents CD161 surface expression (vertical axis, mean fluorescence intensity (MFI)) on polyclonal NK cells incubated for 48 hr at 37° C. in medium supplemented with the indicated concentrations of recombinant human IL-12.

### EXAMPLES

### Example 1

### Construction of LLT1 Multimeric Complexes

Extracellular domains (residues 60-191) (SEQ ID NO: 4) of LLT1 (AF133299, Q9UBP7) (SEQ ID NO: 2) were fused to the human IgG1 FC portion and the chimeric protein produced in transfected eukaryotic cells. The soluble fusion LLT1-Fc protein secreted in the media by the transfected cells was purified on protein-A-Sepharose column and conjugated to proteinA-biotin to generate a multimer. This multimer was labelled with a fluorescent marker using Streptavidin-APC.

This multimer can then be used to visualise interactions between LLT1 and cell surface receptors.

### Method

LLT1 AA sequence (191 amino acid residues)
(AF133299, Q9UHP7):

(SEQ ID NO: 2)

MHDSNNVEKDITPSELPANPGCLHSKEHSIKATLIWRLFFLIMFLTIIVC
GMVAALSAIRANCHQEPSVCLQAACPESWIGFQRKCFYFSDDTKNWTSSQ
RFCDSQDADLAQVESFQELNFLLRYKGPSDHWIGLSREQGQPWKWINGTE
WTRQFPILGAGECAYLNDKGASSARHYTERKWICSKSDIHV
Amino acid residues underlined were removed in the
LLT1-Fc chimeric molecule.

Full length LLT1 cDNA was amplified by reverse transcription (RT)-PCR from polyclonal NK total RNA extracted with TRIzol Reagent (15596-26, Invitrogen) using an oligo(dT) primer for the RT and the primers LLT1-F (5′-ATGCATGACAGTAACAATGTGG-3′) (SEQ ID NO: 9) and LLT1-R (5′-TAGTTGGGGCTTTGCTGTAA-3′) (SEQ ID NO: 10) for the PCR (95° C. for 2 min, 35 cycles with 95° C. for 45 sec, 60° C. for 45 sec, 72° C. for 1 min and a final elongation at 72° C. for 10 min). The PCR product was cloned into pGEMT-easy vector (A1360, Promega).

Codons corresponding to the extracellular domains (60-191) of LLT1 were amplified by PCR using full length LLT1 cDNA as a template and the following primers:

FLLT1BamHI-

(SEQ ID NO: 11)

5′-AAGGGGATCCGAGAGCTAACTGCCATCAAGAGCC-3′
RLLT1NotI-

(SEQ ID NO: 12)

5′-CTCGAGCGGCCGCCTAGACATGTATATCTGATTTGG-3′

The PCR product was subcloned into pcDNA3 vector in frame with the signal sequence of human CD5 and the Fc portion of human IgG1. The final construct was sequenced.

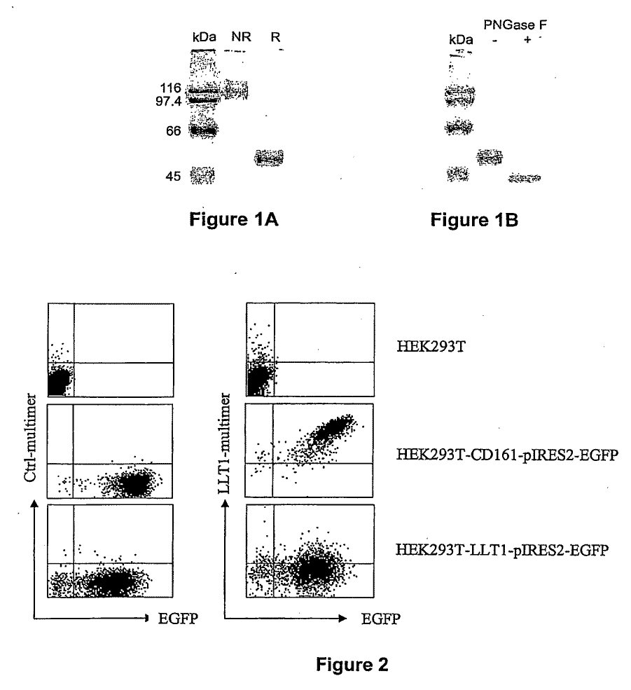

LLT1-Fc dimers were generated by transiently transfecting eukaryotic HEK293T cells. The chimeric protein was released in the media and purified onto a ProteinA-Sepharose column. Purity was assessed by SDS-PAGE electrophoresis (FIG. 1A). Peptide N-glycosidase F (PNGase F) treatment was performed according to the manufacture instruction (New England Biolabs) (FIG. 1B). Briefly, 5 μg of purified LLT1-Fc was denatured in 0.5% SDS, 1% β-mercaptoethanol denaturing buffer at 100° C. for 10 minutes, and digested with 2000 Unit of PNGase F in 1% NP40, 50 mM sodium phosphate pH 7.5 for 1 hour at 37° C.

A multimeric complex was generated by incubating the dimers with ProteinA-biotin (Pierce) at a ratio of 10:1 for 30 minutes at room temperature. Saturating amount of purified human IgG were added to block the remaining free protein A sites. These complexes were conjugated with Streptavidin-APC to generate a fluorescent reagent that can be used in flow cytometry analysis.

A multimer control was generated by incubating ProteinA-biotin with saturing amount of purified human IgG.

### Example 2

### Binding of LLT1 Multimer

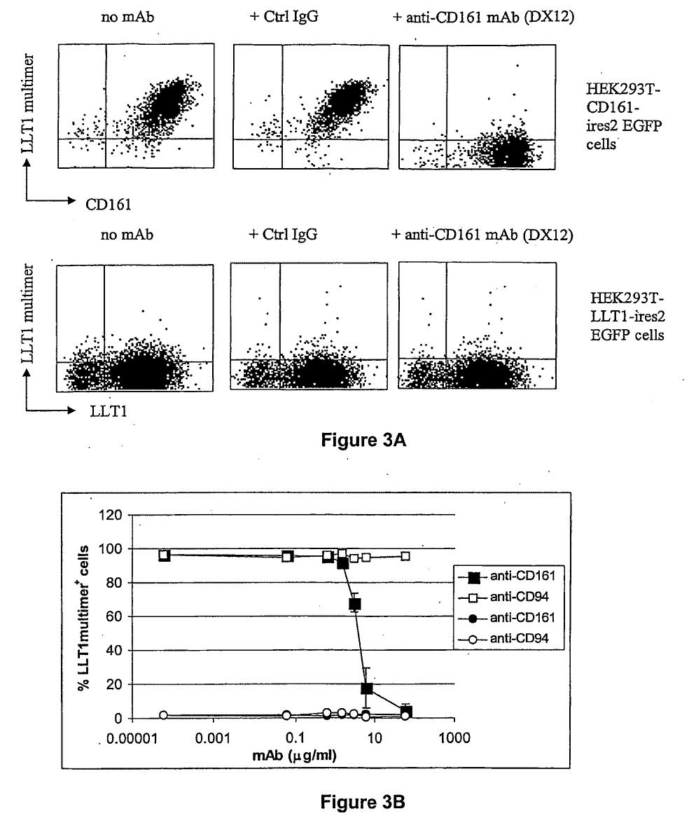

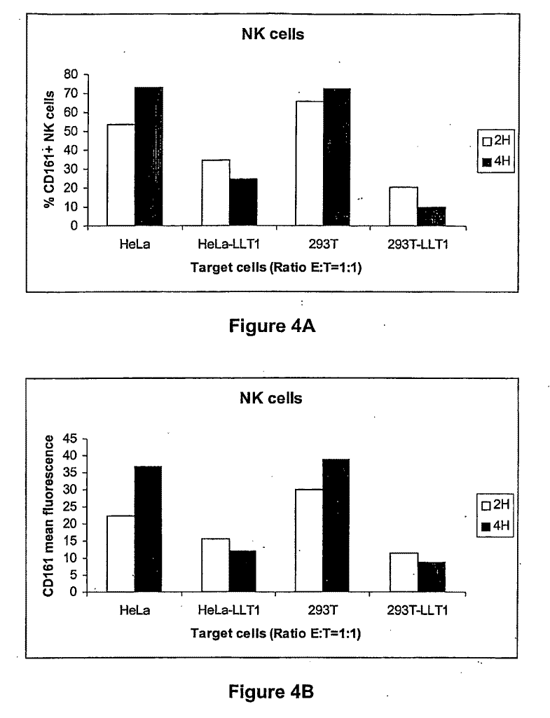

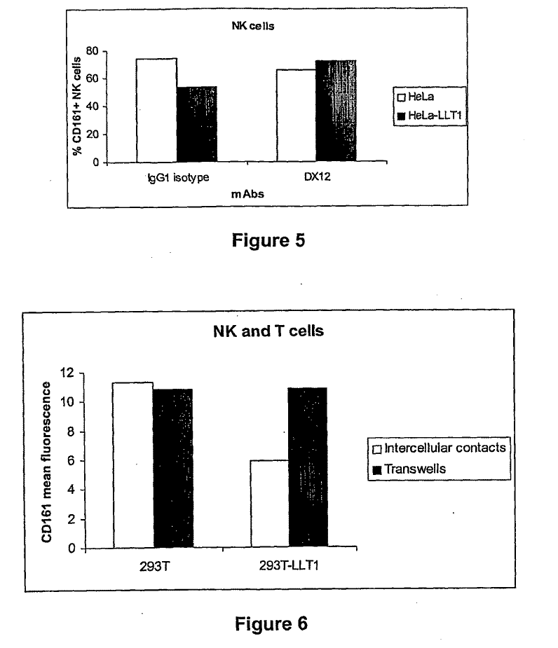

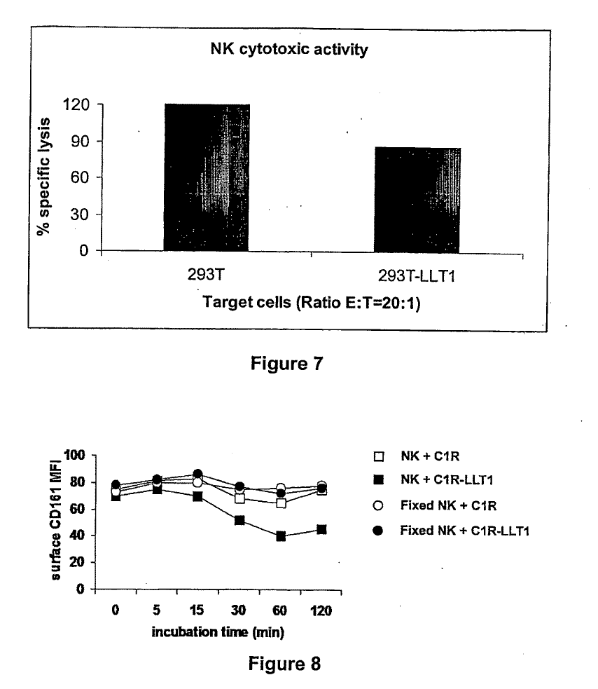

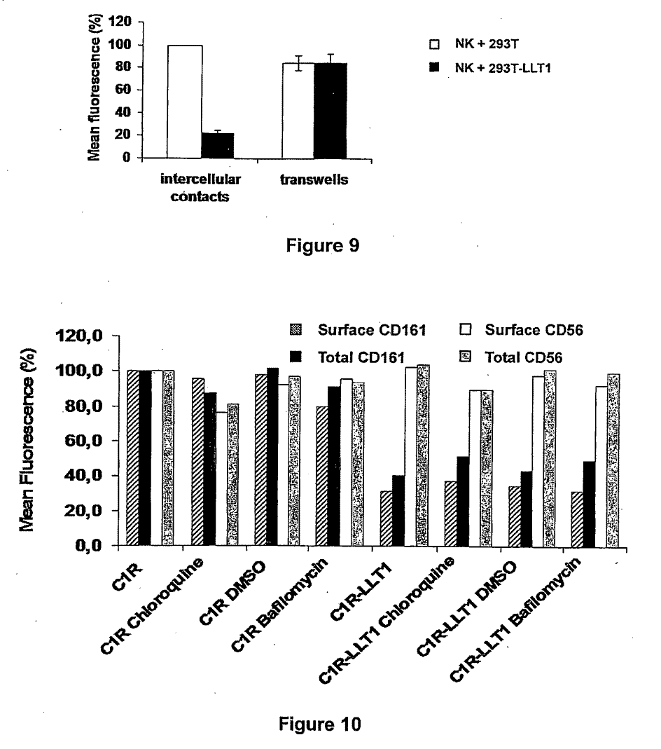

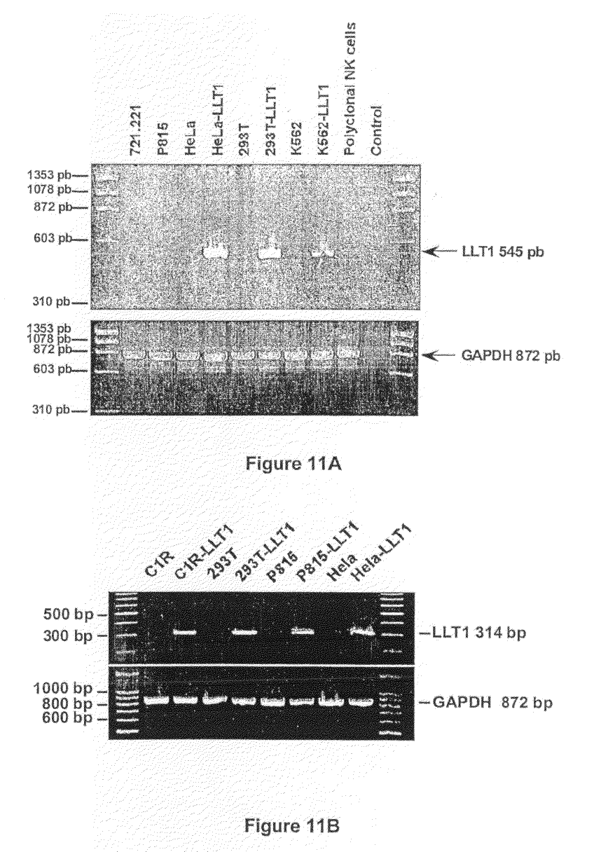

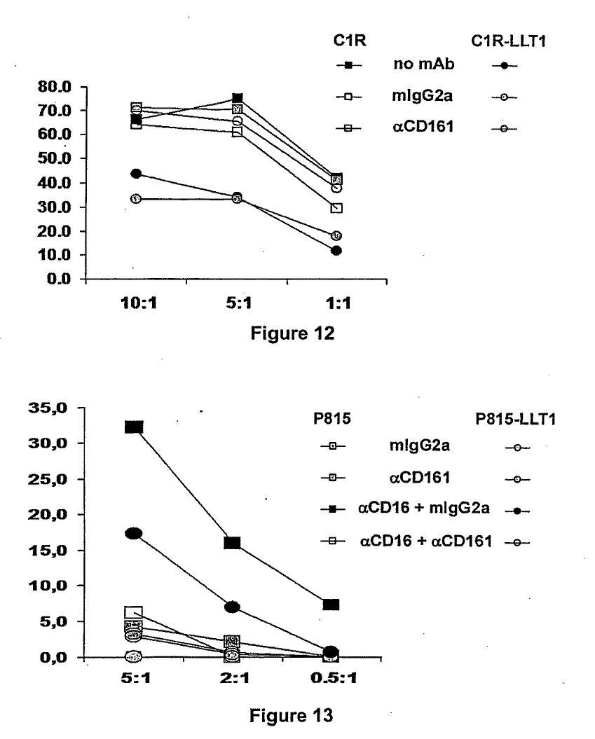

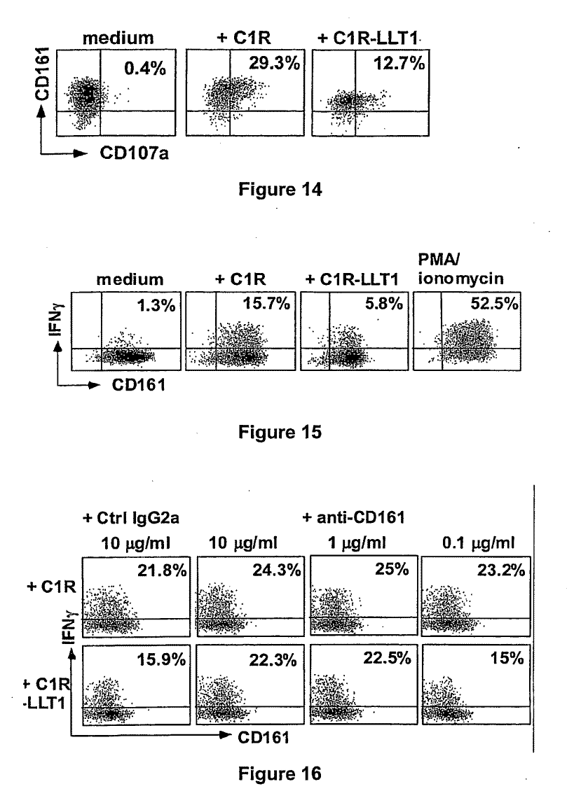

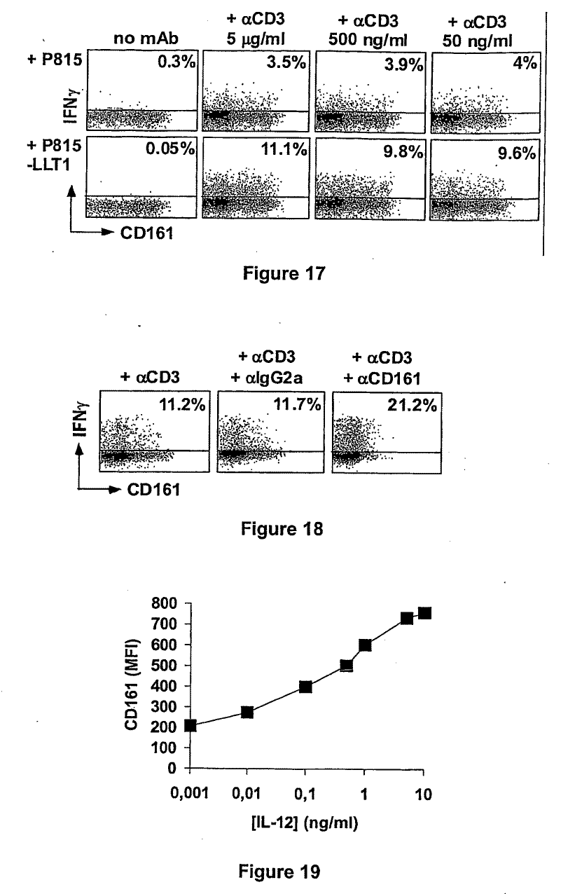

Flow cytometry using the LLT1-Fc-ProteinA-biotin-Streptavidin-APC multimer (LLT1-multimer) was performed to identify the receptor of LLT1. LLT1-multimer stained HEK293T cells stably transfected with human CD161 (111276, Q12918) cloned into pIRES2-EGFP (clontech) while it did not stain untransfected HEK293T or HEK293T stably transfected with LLT1-pIRES2-EGFP (FIG. 2). A control multimer did not stain any of the cells. The specificity of the binding of LLT1-multimer to CD161 was furrier confirmed by addition of anti-CD161 antibodies which abolished the staining while irrelevant isotype control antibodies did not (FIGS. 3A and 3B).

### Example 3

### Interaction of LLT1 with CD161 Induces a Downregulation of CD161

Human polyclonal NK cells were isolated from peripheral mononuclear cells of healthy donors by positive selection using CD56 MicroBeads (Miltenyi Biotec). NK cells were then stimulated with PHA-P (1 ug/ml, SIGMA), irradiated PBMC and B-EBV feeder cells in Iscove's Modification-ATL medium (biowest) supplemented with L-glutamine (2 mM, Gibco), Penicillin-Streptomycin (Gibco) and rIL-2 (500 U/ml).

To assess the role of LLT1 interaction with CD161, various NK cell targets were transfected to express LLT1. The presence of LLT1 transcripts was checked by RT-PCR. No LLT1 transcripts could be detected in 293T, C1R, K562, Hela, 721.221, P815 cell lines but transcripts were detected in their respective LLT1 transfectants (FIGS. 11A, 11B). Furthermore, a LLT1 transcript was detected in an IL-2 activated polyclonal NK cell population and a PHA-Blast population (97% T cells, 3% NK cells (FIG. 1A)).

HEK293T cells, Hela cells and C1R cells, transfected or not with LLT1 were incubated with a polyclonal human NK and T cell population for 5-10 minutes up to 4-5 hours in complete medium at 37° C. Cells were fixed and stained with anti-CD161 antibodies to monitor CD161 level of expression at the cell surface of NK and T cells. A specific and significant loss of CD161⁺ cells is observed when NK and T cells are incubated with cells expressing LLT1 and not with untransfected cells (FIGS. 4A, 4B, and 8). This loss is observed as early as 10 to 15 minutes (FIG. 8) and is abolished by addition of anti-CD161 mAbs (FIG. 5).

This loss of CD161⁺ NK and T cells is not due to release by shedding of a soluble form of LLT1 blocking binding of anti-CD161 antibodies as no effect on CD161 expression was observed when the incubation (4 hours at 37° C.) was performed using transwells (FIGS. 6 and 9), which means that LLT1-induced downregulation of CD161 on NK cells required cell-to-cell contact. This latter result therefore demonstrates that LLT1 induces a downregulation of the CD161 receptor.

Cross-linking of CD161 with anti-CD161 mAb coated onto a plate or bound to FcR-expressing P815 cells could induce some downregulation of CD161.

Experiments using Bafilomycin A1 and chloroquine, which block lysosomal degradation, then suggested that downregulation of CD161 was not due to degradation of the receptor (FIG. 10)

Briefly, surface and total CD161 expression decreased after exposure of NK cells to C1R-LLT1 transfectants in the presence or absence of bafilomycin A1 and chloroquine. NK cells were preincubated with bafilomycin A1 (0.5 μM) and chloroquine (33 μM) for 1 hr at 37° C., followed by incubation for 4 hr with C1R or CLR-LLT1 cells. The mean fluorescence intensities obtained after exposure to negative control C1R cells were arbitrarily set as 100%. CD56 was monitored and used as a negative control marker.

### Example 4

### Expression of LLT1 in 293T Cells Partially Inhibits Polyclonal IL-2 Activated NK Cell-Mediated Cytotoxicity

293T cells transfected or not with LLT1 were labelled with ⁵¹chromium and incubated with polyclonal IL-2 activated NK cells for 18 hours at an E:T ratio of 20:1. Release of ⁵¹chromium was measured in the supernatant and specific lysis of the targets calculated (FIG. 7). A 28.3% lysis decrease was observed for LLT1 expressing cells with respect to control cells.

### Example 5

### Expression of LLT1 on Target Cells Significantly Inhibits Polyclonal IL-2 Activated NK Cell-Mediated Cytotoxicity

NK cell-mediated cytotoxicity of an IL-2 activated polyclonal NK cell population which expressed CD161 on more than 90% of the cells was also measured in a chromium release assay against C1R and C1R-LLT1 target cells. Killing could be restored by addition of blocking anti-CD161 mAb (DX12 or 191B8). Isotype control mIgG2a and anti-CD161 mAb (191B8) were used at 10 μg/ml. LLT1 expression significantly inhibited NK cell-mediated cytotoxicity which was restored with the blocking anti-CD161 mAb (FIG. 12).

Similarly, redirected killing could be inhibited by expression of LLT1 on the FcR expressing cell line P815, or by addition of anti-CD161 mAb to the P815 cell line (see Example 3), together with suboptimal concentration of anti-CD16 (0.5 μg/ml) used to induce NK cell cytotoxicity. Killing could be significantly or completely inhibited by expression of LLT1 on P815 or addition of anti-CD161 mAb (5 μg/ml). (FIG. 13)

In addition, anti-CD161 mAb alone was able to inhibit killing of P815 or P815-LLT1 in assays where polyclonal NK cells showed some cytotoxicity to these targets.

While cytoxicity was assessed in a chromium release assay in the experiments depicted above, inhibition was also measured using a flow cytometry test monitoring CD107a (lamp-1) induction on the NK cell surface when degranulation occurs (FIG. 14). Briefly, polyclonal NK cells were incubated with C1R or C1R-LLT1 cells for 1 hr in the presence of anti-CD107a mAb followed by 4 hr in the presence of Brefeldin A (10 μg/ml) blocking intracellular secretion. Effector to target ratio was 1:5. Cells were fixed and stained for surface expression of CD161.

### Example 6

### LLT1 Interaction with CD161 on NK Cells Inhibits IFN-γ Production

The effect of LLT1 interaction with CD161 on IFN-γ production by NK cells was assessed in a 4 hr assay where polyclonal NK cells were incubated with C1R or C1R-LLT1 target cells in the presence of brefeldin A (10 μg/ml). Effector to target ratio was 1:5. NK cells were identified by cell surface staining using CD3, CD56 and CD161 markers and IFN-γ producing cells by intracellular staining (FIG. 15). A similar assay as above was performed in the presence of isotype control mIgG2a (10 μg/ml) or anti-CD161 191B8 at increasing concentrations (0.1; 1, and 10 μg/ml) (FIG. 16).

Expression of LLT1 significantly blocked IFN-γ production by NK cells. This inhibition was specific as saturating concentrations of blocking anti-CD161 mAb 191B8 could restore IFN-γ production.

### Example 7

### LLT1 Interaction with CD161 on T Cells Enhances CD3-Triggered IFN-γ Production

LLT1 interaction with CD161 induces a downregulation of CD161 on T cells similarly to NK cells. But as opposed to NK cells, LLT1 interaction with CD161 expressed by T cells triggers a costimulatory signal resulting in an increase of IFN-γ production triggered by CD3 ligation (FIGS. 17 and 18).

Briefly, a polyclonal T cell population was incubated for 4 hr at 37° C. with P815 or P815-LLT1 target cells in the presence of increasing concentrations of anti-CD3 and brefeldin A (10 μg/ml). Effector to target ratio was 1:2.5. T cells were identified by cell surface staining using CD3 and CD161 markers and IFN-γ producing cells by intracellular staining (FIG. 17). A polyclonal T cell population was incubated for 4 hr at 37° C. with plate-bound anti-CD3 (500 ng/ml) alone or together with isotype control mIgG2a (5 μg/ml) or anti-CD161 191B8 (5 μg/ml) and in the presence of brefeldin A (10 μg/ml). IFN-γ production by T cells was measured by intracellular staining (FIG. 18).

### Example 8

### IL-12 Upregulates CD161 on NK and T Cells In Vitro

Culture of polyclonal NK cells or T cells in the presence of increasing concentrations of IL-12 upregulates CD161 on their cell surface (FIG. 19). Briefly, polyclonal NK cells were incubated for 48 hr at 37° C. in medium supplemented with the indicated concentrations of recombinant human IL-12.

1-41. (canceled)

42. An isolated compound comprising at least two LLT1 molecules or fragments thereof.

43. An isolated compound according to claim 42, comprising LLT1 fragments as represented by SEQ ID NO: 4.

44. An isolated compound according to claim 42, wherein the LLT1 fragments are linked to anchoring moieties, chosen from a γ-immunoglobulin Fc domain or biotin, and associated together via a linker molecule chosen from protein A or an avidin protein.

45. An isolated compound according to claims 42, wherein said compound also comprises:
toxic or cytolytic moieties, selected from the group consisting of toxins, radioactive molecules, recombinant active molecules or chemicals,
marker moieties, selected from the group consisting of fluorophores, magnetic beads, or haptens.

46. A pharmaceutical composition comprising as active substance, a compound liable, to bind to CD161 or a compound liable to bind to LLT1, in association with a pharmaceutically acceptable carrier.

47. The pharmaceutical composition according to claim 46 comprising as active substance, a mammalian LLT1 or fragments thereof, as compounds liable to bind to CD161, or a mammalian CD161 or fragments thereof, as compounds liable to bind to LLT1, in association with a pharmaceutically acceptable carrier.

48. The pharmaceutical composition according to claim 46, comprising as active substance,
a human LLT1, in particular as represented by SEQ ID NO: 2, or
a fragment of said human LLT1 corresponding to the extracellular domain of LLT1 as represented by SEQ ID NO: 4

49. The pharmaceutical composition according to claim 46, comprising as active substance a compound comprising at least two LLT1 molecules or fragments thereof.

50. The pharmaceutical composition according to claim 46, comprising as active substance:
a human CD161, in particular as represented by SEQ ID NO: 6, or
a fragment of said human CD161 corresponding to the extracellular domain of CD161, (in particular) as represented by SEQ ID NO: 8.

51. The pharmaceutical composition according to claim 46, comprising as active substance an anti-CD161 or anti-LLT1 antibody, a humanized anti-CD161 or anti-LLT1 antibody.

52. A pharmaceutical composition comprising as active substance, single or multiple stranded nucleic acids encoding mammalian, or human, LLT1 or fragments thereof, or their complementary strands in the case of single stranded nucleic acids, as represented respectively by SEQ ID NO: 1 and SEQ ID NO: 3, optionally comprised in a mammalian expression vector, in association with a pharmaceutically acceptable carrier.

53. A pharmaceutical composition comprising as active substance, single or multiple stranded nucleic acids encoding mammalian, or human, CD161 or fragments thereof, or their complementary strands in the case of single stranded nucleic acids, as represented respectively by SEQ ID NO: 5 and SEQ ID NO: 7, optionally comprised in a mammalian expression vector, in association with a pharmaceutically acceptable carrier.

54. A method for the prevention or the treatment of a pathology selected from the group consisting of cancers, infectious diseases, chosen from viral or bacterial, or parasitic diseases, autoimmune diseases, inflammatory diseases, allergic reactions, pregnancy failures, or organ and bone marrow transplantation rejection comprising the administration of a compound of a compound liable to bind to CD161 or a compound liable to bind to LLT1 to a patient in need thereof.

55. The method according to claim 54, of a mammalian LLT1 or fragments thereof, as compounds liable to bind to CD161, or of a mammalian CD161 or fragments thereof, as compounds liable to bind to LLT1.

56. The method according to claim 54, of
a human LLT1, in particular as represented by SEQ ID NO: 2, or
a fragment of said human LLT1 corresponding to the extracellular domain of LLT1 in particular as represented by SEQ ID NO: 4.

57. The method according to claim 54, of a compound comprising at least two LLT1 molecules or fragments thereof.

58. The method according to claim 54, of
a human CD 161, in particular as represented by SEQ ID NO: 6, or
a fragment of said human CD161 corresponding to the extracellular domain of CD 161, in particular as represented by SEQ ID NO: 8.

59. The method according to claim 54, of an anti-CD161 or anti-LLT1 antibody, in particular a humanized anti-CD161 or anti-LLT1 antibody.

60. The method for the prevention or the treatment of a pathology selected from the group consisting of cancers, infectious diseases, chosen from viral or bacterial, or parasitic diseases, autoimmune diseases, inflammatory diseases, allergic reactions, pregnancy failures, or organ and bone marrow transplantation rejection comprising the administration to a patient in need thereof of single or multiple stranded nucleic acids encoding mammalian or human LLT1 or fragments thereof, or their complementary strands in the case of single stranded nucleic acids, as represented respectively by SEQ ID NO: 1 and SEQ ID NO: 3, optionally comprised in a mammalian expression vector.

61. A method for the prevention or the treatment of a pathology selected from the group consisting of cancers, infectious diseases, chosen from viral or bacterial, or parasitic diseases, autoimmune diseases, inflammatory diseases, allergic reactions, pregnancy failures, or organ and bone marrow transplantation rejection comprising the administration to a patient in need thereof of single or multiple stranded nucleic acids encoding mammalian or human CD161 or fragments thereof, or their complementary strands in the case of single stranded nucleic acids as represented respectively by SEQ ID NO: 5 and SEQ ID NO:7, optionally comprised in a mammalian expression vector, in association with a pharmaceutically acceptable carrier.

62. A method for modulating in vitro the activity of cells expressing CD161 and/or LLT1, that wherein said cells are contacted respectively with LLT1 or fragments thereof, and/or with CD161 or fragments thereof.

63. The method according to claim 62, wherein the CD161 expressing cells are NK cells, CD1d restricted Vα24⁺ NKT cells, or T cells, selected from αβ T cells or ys T cells.

64. The method according to claim 62, wherein the CD161 expressing cells are NK cells and the contacting of said cells with LLT1 or fragments thereof leads to the inhibition of said cells.

65. The method according to claim 62, wherein the CD161 expressing cells are T cells or CD1d restricted Vα24⁺ NKT cells and the contacting of said cells with LLT1 or fragments thereof leads to the activation of said cells.

66. The method according to claim 62, wherein the CD161 expressing cells are contacted with:
a human LLT1, in particular as represented by SEQ ID NO: 2, or
a fragment of said human LLT1 corresponding to the extracellular domains of LLT1 as represented by SEQ ID NO: 4.

67. The method according to claim 62, wherein LLT1, or fragments thereof, are presented at the surface of biological membranes, and (in particular) at the surface of cells, are used as soluble molecules, or are immobilized on a support.

68. The method according to claim 62, wherein the LLT1 fragment is part of a compound comprising one or more LLT1 fragments.

69. The method according to claim 62, wherein CD161 expressing cells express the human CD161 receptor, (in particular) as represented by SEQ ID NO: 6.

70. The method according to claim 62, wherein the LLT1 expressing cells are NK cells, T cells, dendritic cells, macrophages, monocytes or B cells.

71. The method according to claim 62, wherein the contacting of LLT1 expressing cells with CD161 or fragments thereof leads to the activation of said cells.

72. The method according to claim 62, wherein the LLT1 expressing cells are contacted with:
a human CD161 as represented by SEQ ID NO: 6, or
a fragment of said human CD161 corresponding to the extracellular domains of CD161, as represented by SEQ ID NO: 8.

73. The method according to claim 62, wherein CD161, or fragments thereof, are presented at the surface of biological membranes, and at the surface of cells, are used as soluble molecules, or are immobilized on a support.

74. The method according to claim 62, wherein the CD161 fragment is part of a compound comprising one or more CD161 fragments.

75. The method according to claim 62, wherein LLT1 expressing cells express the human LLT1 receptor, (in particular) as represented by SEQ ID NO: 2.

76. A method for identifying, purifying, inactivating or destroying CD161 and/or LLT1 expressing cells, wherein said cells are contacted respectively with LLT1 or fragments thereof, and/or with CD161 or fragments thereof.

77. A method for screening compounds liable to be used for the treatment of a pathology selected from the group consisting of cancers, infectious diseases, chosen from viral or bacterial, or parasitic diseases, autoimmune diseases, inflammatory diseases, allergic reactions, pregnancy failures, or organ and bone marrow transplantation rejection, wherein the compounds liable to be used for the treatment of the above mentioned pathologies are selected on their properties of inhibiting the binding between CD161 or fragments thereof, and LLT1 or fragments thereof.

78. A method according to claim 77, wherein the compounds are selected:
on their properties of inhibiting the binding between CD161 or fragments thereof, and LLT1 or fragments thereof.

79. A method according to claim 77, wherein the compounds to screen are antibodies, selected from the group consisting of anti-CD161 or anti-LLT1 antibodies, or fragments thereof, chosen from Fab, F(ab)′₂ or scFv fragments.

80. A method according to claim 79, wherein the antibodies to screen are monoclonal antibodies.

81. A method according to claim 79, wherein the antibodies to screen are humanized antibodies.

82. A method for screening compounds liable to be used for the treatment of a pathology selected from the group consisting of cancers, infectious diseases, chosen from viral or bacterial, or parasitic diseases, autoimmune diseases, inflammatory diseases, allergic reactions, pregnancy failures, or organ and bone marrow transplantation rejection, wherein the compounds liable to be used for the treatment of the above mentioned pathologies are selected on their properties of inducing CD161 internalisation from CD161-expressing cells.

83. A method according to claim 82, wherein the compounds are selected:
on their properties of inducing CD161 internalization from CD161-expressing cells.

84. A method according to claim 82, wherein the compounds to screen are antibodies, selected from the group consisting of anti-CD161 or anti-LLT1 antibodies, or fragments thereof, chosen from Fab, F(ab)₂ or scFv fragments.

85. A method according to claim 84, wherein the antibodies to screen are monoclonal antibodies.

86. A method according to claim 84, wherein the antibodies to screen are humanized antibodies.
